(19) United States
(12) Patent Application Publication
(10) Pub. No.: US 2025/0009623 A1
(43) Pub. Date: Jan. 9, 2025

(54) NOVEL 2,2'-(OCTYLIMINO)BIS[ETHANOL] COMPOSITIONS AND USES THEREOF

(71) Applicant: INTERNATIONAL FLAVORS & FRAGRANCES INC., Union Beach, NJ (US)

(72) Inventors: Allyson Battaglia, Union Beach, NJ (US); Evan Beach, Fanwood, NJ (US); Marie Demazure, Union Beach, NJ (US)

(21) Appl. No.: 18/702,032

(22) PCT Filed: Oct. 19, 2022
(86) PCT No.: PCT/US2022/047168

§ 371 Date: Apr. 17, 2024

Related U.S. Application Data
(60) Provisional application No. 63/257,683, filed on Oct. 20, 2021.

(51) Int. Cl. A61K 8/41; A61L 9/01; A61Q 13/00; A61Q 15/00; C11B 9/00; D06M 13/00

(52) U.S. Cl. CPC A61K 8/41; A61L 9/01; A61Q 13/00; A61Q 15/00; C11B 9/0023; D06M 13/005

(57) ABSTRACT

The present invention pertains to novel 2,2′-(octylimino)bis[ethanol] compositions and their uses in improving fragrance retention and counteracting sweat malodor.

### CROSS-REFERENCE TO RELATED APPLICATION

This application is a 371 of International Application No. PCT/US2022/047168, filed Oct. 19, 2022, which claims priority from U.S. Provisional Application No. 63/257,683, filed Oct. 20, 2021, the contents of which are hereby incorporated by reference in their entirety.

### FIELD OF THE INVENTION

The present invention relates to novel compositions and their uses in providing longevity and durability to fragrance ingredients as well as malodor counteracting effect.

### BACKGROUND OF THE INVENTION

The present invention relates to novel fragrance compositions that exhibit increased fragrance longevity and durability and their uses in personal care products such as deodorants, which possess improved fragrance retention, longer lasting fragrance perception and malodor counteracting effect.

“Malodor” is a term used to describe undesirable or unpleasant odor. Common sources of malodors include body perspiration, smoke, environmental odor such as mold and mildew, bathroom, and etc. Numerous methods have been developed to prevent or reduce malodors in a variety of circumstances. For example, conventional perfumes including a variety of fragrance materials are developed to mask malodors, which generally function via two mechanisms: first, the fragrance materials blend with the malodor compound to provide a different and more desirable aroma; and second, the fragrance materials are employed in a large quantity to overwhelm the malodor compound. However, none of the above malodor counteracting methods provides malodor-specific effect. Consequently, to eliminate the perception of a particular malodor effectively, a high amount of malodor counteracting compositions is often required. Thus, there is a need in the art to provide a malodor-specific counteractant composition that can be effective at a low amount.

The present invention has unexpectedly identified novel 2,2′-(octylimino)bis[ethanol] compositions that provide longevity and durability to certain fragrance ingredients and are selectively effective in counteracting sweat malodor.

### SUMMARY OF THE INVENTION

The present invention is directed to a novel 2,2′-(octylimino)bis[ethanol] composition. When used in a combination with a fragrance compound, 2,2′-(octylimino)bis[ethanol] provides unexpected improvement of fragrance retention and longer lasting fragrance perception.

Specifically, the present invention is directed a fragrance composition comprising 2,2′-(octylimino)bis[ethanol] and a fragrance compound selected from the group consisting of phenethyl alcohol (CAS No. 60-12-8), tetrahydrolinalool (CAS No. 78-69-3), linalool (CAS No. 78-70-6), 3-phenylpropanal (CAS No. 104-53-0), cinnamaldehyde (CAS No. 104-55-2), decanal (CAS No. 112-31-2), ethyl linalool (CAS No. 10339-55-6), dihydromyrcenol (CAS No. 18479-58-8), 2-methyldecanal (19009-56-4), undecavertol (CAS No. 177772-08-6), Veridian (CAS No. 1477699-58-3), and a mixture thereof.

In an embodiment of the present invention, 2,2′-(octylimino)bis[ethanol] and the fragrance compound have a weight ratio of 2:1 to 4:1.

In another embodiment of the present invention, the fragrance compound has a saturated vapor pressure of 0.06 to 0.16 mmHg at 20° C.

In another embodiment of the present invention, the fragrance compound has a logP of 2-5.

In another embodiment of the present invention, the present invention is directed to a method of counteracting sweat malodor in an air space or a substrate comprising the step of introducing a composition into the air space or the substrate, wherein the composition comprises 2,2′-(octylimino)bis[ethanol] or a combination of 2,2′-(octylimino)bis[ethanol] and a fragrance compound selected from the group consisting of phenethyl alcohol, tetrahydrolinalool, linalool, 3-phenylpropanal, cinnamaldehyde, decanal, ethyl linalool, dihydromyrcenol, 2-methyldecanal, undecavertol, Veridian, and a mixture thereof.

These and other embodiments of the present invention will be apparent by reading the following specification.

### BRIEF DESCRIPTION OF THE DRAWINGS

FIG. 1 shows sensory panel intensity ratings (mean±SD; 0—lowest to 10—highest) of sweat malodor in fabric samples treated with an unfragranced fabric refresher spray, the same fabric refresher spray containing fragrance (0.1% Floral HCA), or the fabric refresher spray containing 0.5% of exemplary malodor-counteracting compound 2,2′-(octylimino)bis[ethanol](0.5% C8).

FIG. 2 shows the mean and standard deviation of sweat malodor intensity ratings (0-lowest to 10-highest) reported by subjects of their axilla before (T8h) and after (T12h) a workout session. The “wash-out phase” portion of the graph shows sweat malodor intensity ratings of axilla when subjects used an unfragranced shower gel and no underarm product, which served as a baseline. The “test phase” portion of the graph shows sweat malodor intensity rating of axilla when subjects used the unfragranced shower gel and two underarm products (one to each axilla) with one underarm product being a non-antiperspirant spray (Non-AP Spray) and the other the same Non-AP spray including 0.5% of exemplary malodor-counteracting compound 2,2′-(octylimino)bis[ethanol] (Non-AP Spray+C8 @0.5%).

### DETAILED DESCRIPTION OF THE INVENTION

It has been surprisingly discovered that 2,2′-(octylimino)bis[ethanol] provides improved retention and enhanced perception to certain fragrance compounds. Further, 2,2′-(octylimino)bis[ethanol] compositions provide selective and superior performance in counteracting specific types of malodor such as body perspiration. Accordingly, 2,2′-(octylimino)bis[ethanol] compositions are unexpectedly and advantageously desirable and suitable for uses in a vast number of functional products.

Examples of the functional products are provided herein to illustrate the various aspects of the present invention. However, they do not intend to limit the scope of the present invention. The functional products may include, for example, a conventional room freshener (or deodorant) composition such as room freshener sprays, an aerosol or other spray, fragrance diffusers, a wick or other liquid system, or a solid, for instance candles or a wax base as in pomanders and plastics, powders as in sachets or dry sprays or gels, as in solid gel sticks, clothes deodorants as applied by washing machine applications such as in detergents, powders, liquids, whiteners or fabric softeners, fabric refreshers, linen sprays, closet blocks, closet aerosol sprays, or clothes storage areas or in dry cleaning to overcome residual solvent notes on clothes, bathroom accessories such as paper towels, bathroom tissues, sanitary napkins, towellets, disposable wash cloths, disposable diapers, and diaper pail deodorants, cleansers such as disinfectants and toilet bowl cleaners, cosmetic products such as antiperspirant and deodorants, general body deodorants in the form of powders, aerosols, liquids or solid, feminine care products such as tampons and feminine napkins, baby care products such as diapers, bibs and wipes, or hair care products such as hair sprays, conditioners, rinses, hair colors and dyes, permanent waves, depilatories, hair straighteners, hair groom applications such as pomade, creams and lotions, medicated hair care products containing such ingredients as selenium sulphide, coal tar or salicylates, or shampoos, or foot care products such as foot powders, liquids or colognes, after shaves and body lotions, or soaps and synthetic detergents such as bars, liquids, foams or powders, odor control such as during manufacturing processes, such as in the textile finishing industry and the printing industry (inks and paper), effluent control such as in processes involved in pulping, stock yard and meat processings, sewage treatment, garbage bags, or garbage disposal, or in product odor control as in textile finished goods, rubber finished goods or car fresheners, agricultural and pet care products such as dog and hen house effluents and domestic animal and pet care products such as deodorants, shampoo or cleaning agents, or animal litter material and in large scale closed air systems such as auditoria, and subways and transport systems.

Thus, it will be seen that the composition of the invention is usually one in which the malodor counteractant is present together with a carrier by means of which or from which the malodor counteractant can be introduced into air space wherein the malodor is present, or a substrate on which the malodor has deposited. For example, the carrier can be an aerosol propellant such as a chlorofluoro-methane, or a solid such as a wax, plastics material, rubber, inert powder or gel. Aerosol propellants may be hydrocarbon or halogenated hydrocarbon gas, such as fluorinated hydrocarbons such as 1,1-difluoroethane and/or 1-trifluoro-2-fluoroethane. In some embodiments, the propellant includes liquefied hydrocarbon gases, and C3 to C5 hydrocarbons, including propane, isopropane, butane, isobutane, pentane and isopentane and mixtures of two or more thereof. In some embodiments, the propellants are isobutane, isobutane/isopropane, isobutane/propane and mixtures of isopropane, isobutane and butane. In a wick-type air freshener, the carrier is a substantially odorless liquid of low volatility. In several applications, a composition of the invention contains a surface active agent or a disinfectant, while in others, the malodor counteractant is present on a fibrous substrate. In many compositions of the invention there is also present a fragrance component which imparts a fragrance to the composition. Many types of fragrances can be employed in the present invention, the only limitation being the compatibility with the other components being employed. Suitable fragrances include but are not limited to fruits such as almond, apple, cherry, grape, pear, pineapple, orange, strawberry, raspberry; musk, flower scents such as lavender-like, rose-like, iris-like, carnation-like. Other pleasant scents include herbal and woodland scents derived from pine, spruce and other forest smells. Fragrances may also be derived from various oils, such as essential oils, or from plant materials such as peppermint, spearmint and the like.

A list of suitable fragrances is provided in U.S. Pat. No. 4,534,891, the contents of which are incorporated by reference as if set forth in its entirety. Another source of suitable fragrances is found in Perfumes, Cosmetics and Soaps, Second Edition, edited by W. A. Poucher, 1959. Among the fragrances provided in this treatise are acacia, cassie, chypre, cyclamen, fern, gardenia, hawthorn, heliotrope, honeysuckle, hyacinth, jasmine, lilac, lily, magnolia, mimosa, narcissus, freshly-cut hay, orange blossom, orchid, reseda, sweet pea, trefle, tuberose, vanilla, violet, wallflower, and the like.

In addition to 2,2′-(octylimino)bis[ethanol] and the fragrance compounds of the present invention, other materials can also be used in conjunction with the compositions to encapsulate and/or deliver the compositions. Some well-known materials are, for example, but not limited to, polymers, oligomers, other non-polymers such as surfactants, emulsifiers, lipids including fats, waxes and phospholipids, organic oils, mineral oils, petrolatum, natural oils, perfume fixatives, fibers, starches, sugars and solid surface materials such as zeolite and silica. Some preferred polymers include polyacrylate, polyurea, polyurethane, polyacrylamide, polyester, polyether, polyamide, poly(acrylate-co-acrylamide), starch, silica, gelatin and gum Arabic, alginate, chitosan, polylactide, poly(melamine-formaldehyde), poly(urea-formaldehyde), or a combination thereof.

Malodor counteracting effective amount is understood to mean the amount of the inventive malodor counteractant employed in a functional product that is organoleptically effective to abate a given malodor while reducing the combined intensity of the odor level, wherein the given malodor is present in air space or has deposited on a substrate. The exact amount of malodor counteractant agent employed may vary depending upon the type of malodor counteractant, the type of the carrier employed, and the level of malodor counteractancy desired. In general, the amount of malodor counteractant agent present is the ordinary dosage required to obtain the desired result. Such dosage is known to the skilled practitioner in the art. In a preferred embodiment, when used in conjunction with malodorous solid or liquid functional products, e.g., soap and detergent, the enantiomeric compound-containing composition of the present invention may be present in an amount ranging from about 0.0001% to about 10%, preferably from about 0.001% to about 5%, and more preferably from about 0.01% to about 2%, by weight; and when used in conjunction with malodorous gaseous functional products, the enantiomeric compound-containing composition of the present invention may be present in an amount ranging from about 0.01 to 1 mg per cubic meter of air.

The ratio of 2,2′-(octylimino)bis[ethanol] to fragrance ingredient may be expressed as the ratio of the 2,2′-(octylimino)bis[ethanol] to a single ingredient, regardless of whether there is only one or more fragrance ingredients in the composition. For example, in some embodiments, the ratio of 2,2′-(octylimino)bis[ethanol] to a single fragrance ingredient is at least 1:0.5. In some embodiments, the ratio of 2,2′-(octylimino)bis[ethanol] to a single fragrance ingredient is at least 1:1. In some embodiments, the ratio of 2,2′-(octylimino)bis[ethanol] to a single fragrance ingredient is in a range of about 1:0.5 to 1000:1. In some embodiments, the ratio of 2,2′-(octylimino)bis[ethanol] to a single fragrance ingredient is 100:1, 500:1, or 1000:1. The ratio of the 2,2′-(octylimino)bis[ethanol] may also be expressed as the ratio of the 2,2′-(octylimino)bis[ethanol] to all fragrance ingredients present. For example, in some embodiments, the ratio of 2,2′-(octylimino)bis[ethanol] compound to all fragrance ingredients is in a range of about 1:50 to 10:1. In some embodiments, the ratio of 2,2′-(octylimino)bis[ethanol] to all fragrance ingredients is in a range of about 1:20 to 10:1. In some embodiments, the ratio of 2,2′-(octylimino)bis[ethanol] to all fragrance ingredients is in a range of about 1:10 to 10:1. In some embodiments, the ratio of 2,2′-(octylimino)bis[ethanol] to all fragrance ingredients is in a range of about 1:5 to 10:1. In some embodiments, the ratio of 2,2′-(octylimino)bis[ethanol] to all fragrance ingredients is 1:50, 1:20, 1:10, 1:1, 5:1, or 10:1. In some embodiments, the ratio of 2,2′-(octylimino)bis[ethanol] to all fragrance ingredients is 1:10, 1:5, 1:2, 2:1, 5:1, or 10:1. In some embodiments, the ratio is a weight ratio.

The following are provided as specific embodiments of the present invention. Other modifications of this invention will be readily apparent to those skilled in the art. Such modifications are understood to be within the scope of this invention. All reaction reagents were purchased from Sigma-Aldrich Inc. As used herein all percentages are weight percent unless otherwise noted, ee is understood to be enantiomeric excess, mmHg is understood to be millimeters of mercury, M is understood to be moles per Liter, ppm is understood to stand for parts per million, L is understood to be liter, mL is understood to be milliliter, Kg is understood to be kilogram, and g be gram. IFF as used in the examples is understood to mean International Flavors & Fragrances Inc., New York, NY, USA.

### Example I: Preparation of Test Samples

A series of test solutions comprising (i) 2,2′-(octylimino)bis[ethanol] (“Octyl”) or an analog thereof, methyldiethanolamine (“Methyl”) (CAS 105-59-9) (commercially available from Alfa Aesar Materials Company) or Rewoquat WE 28 E (“Rewo”) (commercially available from Evonik Industries); and (ii) a fragrance compound selected from the group consisting of phenethyl alcohol, tetrahydrolinalool, linalool, 3-phenylpropanal, cinnamaldehyde, decanal, ethyl linalool, dihydromyrcenol, 2-methyldecanal, Veridian, and undecavertol containing a low level of Veridian were prepared in ethanol, wherein 2,2′-(octylimino)bis[ethanol] or an analog thereof was available at a concentration of 14% and the fragrance compound was available at a concentration of 7%.

A series of control solutions containing each of the above fragrance compounds were similarly prepare in ethanol at a concentration of 7%.

### Example II: Improvement of Fragrance Retention

Fragrance retention was evaluated by the quantitative determination of the fragrance compounds in space, which was conducted using headspace technique, total ion count mass spectrometry.

Testing Procedure: The test samples of EXAMPLE I (10 mL) were each deposited onto a 1″ blotter paper strip resting in a precleaned 20 mL vial for volatile organic compounds (VOC) sampling. Three replicate samples were prepared for each test sample including the control solutions. Vials were then stored at 37° C. in a ventilated oven for 3-6 hours. The vials were capped and equilibrated to room temperature. Headspace was collected onto Gerstel Tenax-TA tubes, desorbed using the GERSTEL Thermal Desorption Unit (TDU), and analyzed with gas chromatography-mass spectrometry (GC-MS).

Results and Discussion: Average of total ion count (TIC) and standard deviation (SD) of the test samples are reported in the following. The TIC ratios of 2,2′-(octylimino)bis[ethanol] (“Octyl”), methyldiethanolamine (“Methyl”) and Rewoquat® WE 28 E (“Rewo”) test solutions and the control solutions are represented as RO, RM and RR, respectively.

| | | Headspace Intensity (×1000) (TIC ± SD) | | | | | | |
|---|---|---|---|---|---|---|---|---|
| | Fragrance | Control | Octyl | RO | Methyl | RM | Rewo | RR |
| 1 | Phenethyl Alcohol | 4753 ± 1140 | 30205 ± 3363 | 6.35 | 23673 ± 934 | 4.98 | 20202 ± 1784 | 4.25 |
| 2 | Tetrahydrolinalool | 2142 ± 802 | 31294 ± 7532 | 14.61 | 4560 ± 308 | 2.13 | 5814 ± 46100 | 2.71 |
| 3 | Linalool | 4825 ± 3168 | 58848 ± 25517 | 12.2 | 11615 ± 2173 | 2.41 | 15232 ± 11760 | 3.16 |
| 4 | 3-Phenylpropanal | 3744 ± 1745 | 15488 ± 3359 | 4.14 | 7094 ± 1077 | 1.89 | 9260 ± 3123 | 2.47 |
| 5 | Cinnamaldehyde | 4554 ± 450 | 25545 ± 3065 | 5.61 | 12429 ± 3409 | 2.73 | 16335 ± 222 | 3.59 |
| 6 | Decanal | 915 ± 63 | 5654 ± 3657 | 6.18 | 1338 ± 375 | 1.46 | 1138 ± 283 | 1.24 |
| 7 | Ethyl Linalool | 1433 ± 610 | 22231 ± 4981 | 15.51 | 7994 ± 2065 | 5.58 | 5153 ± 4200 | 3.6 |
| 8 | Dihydromyrcenol | 5105 ± 1071 | 61187 ± 5955 | 11.99 | 11518 ± 4355 | 2.26 | 11966 ± 8256 | 2.34 |
| 9 | 2-Methyldecanal | 490 ± 104 | 13681 ± 4752 | 27.92 | 1517 ± 212 | 3.1 | 1869 ± 714 | 3.82 |
| 10 | Veridian | 1426 ± 257 | 5167 ± 803 | 3.62 | 2360 ± 723 | 1.65 | 1145 ± 563 | 0.8 |
| 11 | Undecavertol | 236 ± 97 | 2827 ± 1512 | 11.97 | 863 ± 214 | 3.65 | 765 ± 498 | 3.24 |

Among all the test samples, 2,2′-(octylimino)bis[ethanol] provided higher TIC when compared with methyldiethanolamine and Rewoquat® WE 28 E. In particular, 2,2′-(octylimino)bis[ethanol] exhibited superior performance when combined with tetrahydrolinalool, linalool, decanal, ethyl linalool, dihydromyrcenol, 2-methyldecanal or undecavertol.

### Example III: Unexpected Selectivity of 2,2′-(Octylimino)bis[ethanol] Compositions

The improvement of fragrance retention provided by 2,2′-(octylimino)bis[ethanol] is surprising and unexpected because such an effect depends on selective fragrance compounds. 2,2′-(Octylimino)bis[ethanol] does not provide prolonged retention to all fragrance compounds.

For example, 2,2′-(octylimino)bis[ethanol] failed to achieve prolonged fragrance retention when combined with Delphone (CAS No. 4819-67-4), Verdox (CAS No. 88-41-5), Floriffol (CAS No. 185019-16-3), Allyl Amyl Glycolate (CAS No. 124899-75-8), dimethyl benzyl carbinyl acetate (CAS No. 151-05-3), Delta Damascone (CAS No. 57378-68-4), Styralyl acetate (CAS No. 93-92-5), dimethyl octanol (CAS No. 106-21-8), fleuramone (CAS No. 137-03-1), geranyl acetate (CAS No. 105-87-3), Aupebine (CAS No. 123-11-5), Citronellol 950 (CAS No. 106-22-9), alpha-isomethyl ionone (CAS No. 127-51-5), Cashmeran (CAS No. 33704-61-9), Geraniol 980 Pure (CAS No. 106-24-1), beta-ionon (CAS No. 14901-07-6), Amber Xtreme and Calone (CAS No. 28940-11-6).

### Example IV: Evaluation of 2,2′-(Octylimino)bis[ethanol] and Fragrance Compound Combinations at Different Ratios

A series of solutions containing 2,2′-(octylimino)bis[ethanol] (“Octyl”) and the fragrance compound dihydromyrcenol (“DHM”) at different ratios were prepared similarly according to EXAMPLE I and evaluated for fragrance retention properties using the TIC mass spectrometry. The average of TIC and SD as well as the TIC ratios of the test and control samples (RO) are reported in the following.

| Ratio (Octyl:DHM) | Headspace Intensity (x1000) (TIC ± SD) | RO |
|---|---|---|
| Control | 3674 ± 855 | N/A |
| 1:4 | 3548 ± 352 | 0.97 |
| 1:2 | 4568 ± 731 | 1.24 |
| 1:1 | 11823 ± 2192 | 3.22 |
| 2:1 | 51514 ± 15526 | 14.02 |
| 4:1 | 64099 ± 5535 | 17.45 |
| 5:1 | 49089 ± 3127 | 13.36 |
| 6:1 | 41558 ± 5357 | 11.31 |
| 7:1 | 40645 ± 2927 | 11.06 |

It was surprisingly found that only combinations of 2,2′-(octylimino)bis[ethanol] and dihydromyrcenol having a weight ratio of at least 1:1 and preferably from 2:1 to 4:1 exhibited superior performance.

### Example V

Establishment of Malodor Models: The sweat, bathroom, mildew, and smoke malodor models were prepared based on Applicants' proprietary formulations for assessing the effectiveness of various malodor counteractants.

Preparation of Test Samples: Samples of mixtures of a malodor material and (i) various test compounds including 2,2′-(octylimino)bis[ethanol] (“Octyl”), methyldiethanolamine (“Methyl”), 2,2′-(butylimino)bis[ethanol] (“Butyl”) (CAS No. 102-79-4), 2,2′-(dodecylimino)bis[ethanol] (“Dodecyl”) (CAS No. 1541-67-9) and 2,2′-(octadecylimino)bis[ethanol] (“Octadecyl”) (CAS No. 10213-78-2) diluted in a solvent (0.5%), respectively; or (ii) a solvent alone control were pipetted into plastic pellets and placed in plastic squeeze bottles. The bottles were capped and the samples were allowed to equilibrate for one hour before the testing.

Testing Procedure: 24 Trained panelists (consisting of men/women with an age range of 25 to 55). The panelists were instructed to take the steps of i) uncap a bottle; ii) place their noses at a distance of about 3-4 inches above the opening; iii) take short sniffs for 3 seconds while squeezing the bottle; and v) enter a rating of overall intensity and malodor intensity on a handheld computer.

The overall and malodor intensity was rated using the Labeled Magnitude Scale (LMS) [Green, et al., Chemical Senses, 21(3), Jun 1996, 323-334]. Percent malodor reduction (“% MOR”) represents the perceived reduction in mean malodor intensity of the sample containing the malodor in the presence of a malodor counteractant relative to the negative control (Malodor Alone).

Results and Discussion: The mean ranks of the malodor coverage for the above test were as follows:

| | % MOR | | | | |
|---|---|---|---|---|---|
| | Octyl | Methyl | Butyl | Dodecyl | Octadecyl |
| Sweat | 66 | 55 | 48 | 51 | 32 |
| Bathroom | 21 | 10 | 27 | 21 | 9 |
| Mildew | 17 | 14 | 5 | 5 | 7 |
| Smoke | 6 | 4 | 7 | 2 | 0 |

When compared with its analogs, 2,2′-(Octylimino)bis[ethanol] of the present invention was confirmed to be significantly and specifically effective in counteracting sweat malodor.

### Example VI: Evaluation of 2,2′-(alkylimino)bis[ethanol]s Having Different Alkyl Chain Lengths on Aldehyde Stability

To assess the impact of 2,2′-(alkylimino)bis[ethanol]s having different alkyl chain lengths on the stability of aldehydes, a fragrance accord containing equal proportions by weight of octanal, nonanal, decanal, 10-undecenal, and dodecanal was prepared. Individual test samples were prepared by mixing the fragrance accord with one 2,2′-(alkylimino)bis[ethanol] having either a C₄, C₈, C₁₂, or C₁₈ alkyl chain length at a weight ratio of 1:2 (2,2′-(alkylimino)bis[ethanol]:fragrance accord). Control samples contained the fragrance ingredient without any 2,2′-(alkylimino)bis[ethanol]s. All samples were prepared in triplicate and stored at 40° C. for 4 weeks. No efforts were made to exclude air from the samples. Immediately prior to analysis by GC-MS, samples were diluted with acetone to give 1% w/w fragrance accord. GC peak areas are reported as total ion count (TIC) in TABLE E4 and the difference relative to control is shown in TABLE E5. The 2,2′-(alkylimino)bis[ethanol] tested is denoted by the alkyl chain length (C4, C8, C12, C18) and the control is denoted by “Control” in the tables.

TABLE E4
TIC (mean ± standard deviation) for each test and control sample.

| | Control | C4 | C8 | C12 | C18 |
|---|---|---|---|---|---|
| Octanal | 7.55E+07 ± | 2.70E+07 ± | 2.66E+07 ± | 2.39E+07 ± | 2.05E+07 ± |
| | 7.27E+06 | 2.08E+05 | 2.38E+06 | 1.32E+06 | 9.57E+05 |
| Nonanal | 8.65E+07 ± | 3.34E+07 ± | 3.26E+07 ± | 2.98E+07 ± | 2.55E+07 ± |
| | 9.65E+06 | 4.55E+05 | 3.21E+06 | 1.63E+06 | 1.15E+06 |
| Decanal | 9.02E+07 ± | 3.85E+07 ± | 3.80E+07 ± | 3.52E+07 ± | 2.99E+07 ± |
| | 1.06E+07 | 5.64E+05 | 3.76E+06 | 1.86E+06 | 1.18E+06 |
| 10-undecanal | 9.90E+07 ± | 4.59E+07 ± | 4.52E+07 ± | 4.15E+07 ± | 3.51E+07 ± |
| (Ulenic) | 1.24E+07 | 7.71E+05 | 4.23E+06 | 1.40E+06 | 1.15E+06 |
| Dodecanal (Lauric) | 9.62E+07 ± | 5.36E+07 ± | 5.08E+07 ± | 4.62E+07 ± | 3.85E+07 ± |
| | 1.20E+07 | 2.89E+05 | 4.82E+06 | 1.15E+06 | 1.18E+06 |

TABLE E5
Percent change in TIC relative to control.

| | C4 | C8 | C12 | C18 |
|---|---|---|---|---|
| Octanal | −64% | −65% | −68% | −73% |
| Nonanal | −61% | −62% | −66% | −71% |
| Decanal | −57% | −58% | −61% | −67% |
| 10-undecanal (Ulenic) | −54% | −54% | −58% | −65% |
| Dodecanal (Lauric) | −44% | −47% | −52% | −60% |

These results suggest, unexpectedly, that 2,2′-(alkylimino)bis[ethanol]s having shorter alkyl chain lengths have less impact on aldehyde stability compared to those with longer chain lengths. These results are supportive of the use of 2,2′-(alkylimino)bis[ethanol] having shorter alkyl chain lengths (e.g., C₄₋₁₂) in fragrance compositions containing aldehydes.

### Example VII: Evaluation of 2,2′-(alkylimino)bis[ethanol]s Having Different Alkyl Chain Lengths on the Stability of Ester Fragrance Ingredients

To assess the impact of 2,2′-(alkylimino)bis[ethanol]s having different alkyl chain lengths on the stability of ester fragrance ingredients, three fragrance accords were created. The first fragrance accord contained equal proportions by weight of cis-3-hexenyl acetate, benzyl acetate, geranyl acetate, methyl dihydrojasmonate, hexyl salicylate, (E)-oxacycloheptadec-10-en-2-one (ambrettolide), and ethylene brassylate was prepared. The second fragrance accord contained equal proportions by weight of prenyl acetate, allyl caproate, allyl heptanoate, allyl amyl glycolate, linalyl acetate, allyl caprylate, pinocarvyl acetate, ethyl linalyl acetate, neryl acetate, geranyl acetate, linalyl isobutyrate, cyclogalbanate, allyl cyclohexyl propionate, dihydrocuminyl acetate, cinnamyl acetate, allyl phenoxyacetate, geranyl propionate, geranyl isobutyrate, geranyl tiglate, and farnesyl acetate. The third fragrance accord contained equal proportions by weight of p-cresyl acetate, methyl salicylate, ethyl salicylate, Honey F, p-cresyl isobutyrate, methyl anisate, coumarin, isobutyl salicylate, maltol isobutyrate, eugenyl acetate, amyl salicylate, Celeriax, Oceanol, Iso Butavan, cis-3-hexenyl salicylate, hexyl salicylate, Veramoss, cyclohexyl salicylate, and phenethyl salicylate. In the third fragrance accord, the amyl salicylate was a mixture of 2-methylbutyl and n-amyl esters in approximately a 1:2 ratio.

Individual test samples were prepared by mixing each of the fragrance accords with one 2,2′-(alkylimino)bis[ethanol] having either a C₁, C₄, C₅, or C₁₂ alkyl chain length at a weight ratio of 1:2 (2,2′-(alkylimino)bis[ethanol]:fragrance accord). Control samples contained the fragrance accord without any 2,2′-(alkylimino)bis[ethanol]s. All samples were prepared in triplicate and stored at 40° C. for 4 weeks. No efforts were made to exclude air from the samples.

Immediately prior to analysis by GC-MS, samples were diluted with acetone to give 1% w/w fragrance accord. GC peak areas are reported as total ion count (TIC) in TABLE E6 and the difference relative to control is shown in TABLE E7. In the second fragrance accord, it should be appreciated that geranyl propionate contains about 25% of the corresponding citronellyl ester, thus the additional peak is noted in TABLES E6-E7. The 2,2′-(alkylimino)bis[ethanol] tested is denoted by the alkyl chain length (C1, C4, C8, C12) and the control is denoted by “Control” in the tables.

TABLE E6
TIC (mean ± standard deviation) for test and control samples of each fragrance accord.
Isomer peaks for an ingredient are denoted by “i” and “ii”.

| | | | | |
|---|---|---|---|---|
| Control | C1 | C4 | C8 | C12 |

FIRST FRAGRANCE ACCORD

| | | | | | |
|---|---|---|---|---|---|
| (E)- | 8.49E+07 ± | 7.02E+07 ± | 7.73E+07 ± | 8.11E+07 ± | 7.98E+07 ± |
| oxacycloheptadec-10- | 3.82E+06 | 5.59E+06 | 6.86E+06 | 3.77E+06 | 1.62E+06 |
| en-2-one | | | | | |
| Benz Acet | 8.33E+07 ± | 2.33E+07 ± | 3.97E+07 ± | 5.58E+07 ± | 6.25E+07 ± |
| | 4.33E+06 | 1.93E+06 | 3.83E+06 | 2.23E+06 | 1.05E+06 |
| Cis-3-Hexenyl Acet | 6.60E+07 ± | 4.19E+07 ± | 5.22E+07 ± | 5.97E+07 ± | 6.25E+07 ± |
| | 3.20E+06 | 3.76E+06 | 4.97E+06 | 2.58E+06 | 1.28E+06 |
| Ethylene Brassylate | 8.30E+07 ± | 3.44E+07 ± | 5.26E+07 ± | 6.48E+07 ± | 7.02E+07 ± |
| | 4.00E+06 | 3.00E+06 | 4.77E+06 | 3.09E+06 | 1.80E+06 |
| Geranyl Acet | 8.08E+07 ± | 4.98E+07 ± | 6.10E+07 ± | 7.08E+07 ± | 7.36E+07 ± |
| | 4.26E+06 | 4.35E+06 | 5.99E+06 | 3.39E+06 | 1.89E+06 |
| Hexyl Sal | 8.84E+07 ± | 4.11E+07 ± | 6.13E+07 ± | 7.23E+07 ± | 7.56E+07 ± |
| | 5.10E+06 | 3.80E+06 | 5.88E+06 | 3.84E+06 | 2.10E+06 |
| Meth DH Jasmonate i | 7.38E+07 ± | 4.83E+07 ± | 5.91E+07 ± | 6.67E+07 ± | 6.76E+07 ± |
| | 3.71E+06 | 4.10E+06 | 5.51E+06 | 3.00E+06 | 1.42E+06 |
| Meth DH Jasmonate | 7.94E+06 ± | 4.13E+06 ± | 5.34E+06 ± | 6.13E+06 ± | 6.27E+06 ± |
| ii | 3.52E+05 | 3.46E+05 | 4.77E+05 | 2.48E+05 | 1.22E+05 |

SECOND FRAGRANCE ACCORD

| | | | | | |
|---|---|---|---|---|---|
| Allyl Amyl Glycolate | 2.34E+07 ± | 6.57E+06 ± | 5.73E+06 ± | 6.21E+06 ± | 7.62E+06 ± |
| | 1.12E+06 | 2.98E+05 | 3.60E+05 | 5.54E+05 | 8.50E+05 |
| Allyl Caproate | 2.31E+07 ± | 1.61E+07 ± | 1.87E+07 ± | 2.10E+07 ± | 2.14E+07 ± |
| | 1.08E+06 | 8.44E+05 | 1.40E+06 | 1.31E+06 | 2.66E+06 |
| Allyl Caprylate | 2.60E+07 ± | 1.87E+07 ± | 2.10E+07 ± | 2.32E+07 ± | 2.37E+07 ± |
| | 1.16E+06 | 8.94E+05 | 1.51E+06 | 1.53E+06 | 2.74E+06 |
| Citronellyl Prop | 8.36E+06 ± | 8.27E+06 ± | 7.94E+06 ± | 7.92E+06 ± | 7.80E+06 ± |
| | 3.52E+05 | 4.35E+05 | 7.54E+05 | 5.23E+05 | 8.75E+05 |
| Cyclogalbanate | 2.68E+07 ± | 3.00E+06 ± | 3.95E+06 ± | 6.02E+06 ± | 7.93E+06 ± |
| | 1.31E+06 | 1.14E+05 | 2.79E+05 | 6.48E+05 | 8.67E+05 |
| Eth Linalyl Acet i | 1.30E+07 ± | 1.43E+07 ± | 1.22E+07 ± | 1.26E+07 ± | 1.26E+07 ± |
| | 6.59E+05 | 6.15E+05 | 9.84E+05 | 8.55E+05 | 1.50E+06 |
| Eth Linalyl Acet ii | 1.98E+07 ± | 1.88E+07 ± | 1.86E+07 ± | 1.93E+07 ± | 1.92E+07 ± |
| | 9.84E+05 | 9.85E+05 | 1.16E+06 | 1.27E+06 | 2.35E+06 |
| Farnesyl Acet i | 9.25E+06 ± | 6.01E+06 ± | 7.15E+06 ± | 7.53E+06 ± | 7.95E+06 ± |
| | 4.58E+05 | 2.99E+05 | 4.92E+05 | 5.74E+05 | 9.69E+05 |
| Farnesyl Acet ii | 1.50E+07 ± | 9.66E+06 ± | 1.16E+07 ± | 1.31E+07 ± | 1.37E+07 ± |
| | 7.79E+05 | 5.24E+05 | 7.91E+05 | 1.00E+06 | 1.48E+06 |
| Geranyl Acet | 4.24E+07 ± | 2.52E+07 ± | 3.13E+07 ± | 3.57E+07 ± | 3.69E+07 ± |
| | 2.03E+06 | 1.28E+06 | 2.28E+06 | 2.75E+06 | 4.72E+06 |
| Geranyl Iso Buty | 1.77E+07 ± | 1.57E+07 ± | 1.62E+07 ± | 1.68E+07 ± | 1.67E+07 ± |
| | 8.84E+05 | 7.54E+05 | 1.06E+06 | 1.17E+06 | 2.01E+06 |
| Geranyl Prop | 1.63E+07 ± | 1.21E+07 ± | 1.36E+07 ± | 1.47E+07 ± | 1.49E+07 ± |
| | 8.18E+05 | 6.00E+05 | 8.71E+05 | 1.04E+06 | 1.73E+06 |
| Geranyl Tiglate | 3.06E+07 ± | 2.84E+07 ± | 2.86E+07 ± | 2.93E+07 ± | 2.90E+07 ± |
| | 1.66E+06 | 1.58E+06 | 1.71E+06 | 2.22E+06 | 3.36E+06 |
| Linalyl Acet | 3.26E+07 ± | 3.10E+07 ± | 3.04E+07 ± | 3.19E+07 ± | 3.18E+07 ± |
| | 1.67E+06 | 1.59E+06 | 1.97E+06 | 2.03E+06 | 3.90E+06 |
| Linalyl Iso Buty | 3.17E+07 ± | 3.17E+07 ± | 3.09E+07 ± | 3.15E+07 ± | 3.11E+07 ± |
| | 1.71E+06 | 1.68E+06 | 1.67E+06 | 2.18E+06 | 3.58E+06 |
| Neryl Acet | 1.80E+07 ± | 1.17E+07 ± | 1.40E+07 ± | 1.56E+07 ± | 1.61E+07 ± |
| | 8.68E+05 | 5.76E+05 | 9.77E+05 | 1.09E+06 | 1.88E+06 |
| Pinocarvyl Acet | 3.07E+07 ± | 2.82E+07 ± | 2.83E+07 ± | 2.98E+07 ± | 2.97E+07 ± |
| | 1.40E+06 | 1.29E+06 | 1.84E+06 | 1.81E+06 | 3.37E+06 |
| Prenyl Acet | 2.17E+07 ± | 1.28E+07 ± | 1.62E+07 ± | 1.90E+07 ± | 1.97E+07 ± |
| | 1.11E+06 | 7.59E+05 | 1.24E+06 | 1.31E+06 | 2.60E+06 |

THIRD FRAGRANCE ACCORD

| | | | | | |
|---|---|---|---|---|---|
| Amyl Sal | 2.24E+07 ± | 1.10E+07 ± | 1.57E+07 ± | 1.85E+07 ± | 2.07E+07 ± |
| | 7.90E+05 | 5.86E+04 | 6.12E+05 | 9.08E+05 | 1.71E+06 |
| Celeriax i | 2.65E+07 ± | Not detected | Not detected | 6.17E+05 ± | 3.68E+06 ± |
| | 8.10E+05 | | | 1.07E+06 | 2.72E+05 |
| Celeriax ii | 2.08E+06 ± | Not detected | Not detected | Not detected | Not detected |
| | 6.55E+04 | | | | |
| Cis-3-Hexenyl Sal | 2.98E+07 ± | 1.10E+07 ± | 1.72E+07 ± | 2.24E+07 ± | 2.61E+07 ± |
| | 9.01E+05 | 1.40E+05 | 7.08E+05 | 1.11E+06 | 2.17E+06 |
| Coumarin | 2.61E+07 ± | 2.43E+07 ± | 2.46E+07 ± | 2.47E+07 ± | 2.56E+07 ± |
| | 9.77E+05 | 4.94E+05 | 8.76E+05 | 1.22E+06 | 1.94E+06 |
| Cresyl Acet | 3.28E+07 ± | Not detected | Not detected | Not detected | Not detected |
| | 9.49E+05 | | | | |
| Cresyl Iso Buty | 3.32E+07 ± | Not detected | Not detected | Not detected | Not detected |
| | 8.63E+05 | | | | |
| Cyclohexyl Sal | 3.33E+07 ± | 2.92E+07 ± | 3.17E+07 ± | 3.13E+07 ± | 3.24E+07 ± |
| | 1.09E+06 | 6.34E+05 | 1.14E+06 | 1.58E+06 | 2.57E+06 |
| Eth Sal | 3.40E+07 ± | 1.36E+07 ± | 2.10E+07 ± | 2.65E+07 ± | 3.04E+07 ± |
| | 1.03E+06 | 2.56E+05 | 8.36E+05 | 1.39E+06 | 2.09E+06 |
| Hexyl Sal | 3.36E+07 ± | 1.73E+07 ± | 2.39E+07 ± | 2.78E+07 ± | 3.08E+07 ± |
| | 1.07E+06 | 2.26E+05 | 8.92E+05 | 1.32E+06 | 2.51E+06 |
| Honey Fi | 8.08E+06 ± | Not detected | Not detected | Not detected | Not detected |
| | 2.29E+05 | | | | |
| Honey Fii | 2.17E+07 ± | Not detected | Not detected | Not detected | Not detected |
| | 6.41E+05 | | | | |
| Iso Amyl Sal | 1.15E+07 ± | 8.66E+06 ± | 1.14E+07 ± | 1.22E+07 ± | 1.26E+07 ± |
| | 3.50E+05 | 1.51E+05 | 4.24E+05 | 5.76E+05 | 9.22E+05 |
| Iso Butavan | 2.38E+07 ± | Not detected | Not detected | Not detected | Not detected |
| | 7.31E+05 | | | | |
| Maltol Iso Buty | 1.83E+07 ± | Not detected | Not detected | Not detected | 1.83E+06 ± |
| | 7.05E+05 | | | | 2.18E+05 |
| Meth Anisate | 3.28E+07 ± | 2.72E+07 ± | 2.94E+07 ± | 3.04E+07 ± | 3.19E+07 ± |
| | 1.07E+06 | 4.76E+05 | 1.05E+06 | 1.38E+06 | 2.20E+06 |
| Meth Sal | 3.05E+07 ± | 1.07E+07 ± | 1.05E+07 ± | 1.52E+07 ± | 2.12E+07 ± |
| | 9.79E+05 | 2.32E+05 | 4.12E+05 | 7.91E+05 | 1.52E+06 |
| Oceanol | 2.38E+07 ± | 2.06E+07 ± | 2.22E+07 ± | 2.25E+07 ± | 2.37E+07 ± |
| | 7.85E+05 | 4.71E+05 | 8.53E+05 | 1.65E+06 | 2.48E+06 |

TABLE E7
Percent change in TIC relative to control for test samples
of each fragrance accord.

| | | | |
|---|---|---|---|
| C1 | C4 | C8 | C12 |

FIRST FRAGRANCE ACCORD

| | | | | |
|---|---|---|---|---|
| (E)-oxacycloheptadec-10-en-2- | −17% | −9% | −4% | −6% |
| one | | | | |
| Benz Acet | −72% | −52% | −33% | −25% |
| Cis-3-Hexenyl Acet | −36% | −21% | −9% | −5% |
| Ethylene Brassylate | −59% | −37% | −22% | −15% |
| Geranyl Acet | −38% | −25% | −12% | −9% |
| Hexyl Sal | −53% | −31% | −18% | −14% |
| Meth DH Jasmonate i | −35% | −20% | −10% | −8% |
| Meth DH Jasmonate ii | −48% | −33% | −23% | −21% |

SECOND FRAGRANCE ACCORD

| | | | | |
|---|---|---|---|---|
| Allyl Amyl Glycolate | −72% | −76% | −74% | −67% |
| Allyl Caproate | −31% | −19% | −9% | −8% |
| Allyl Caprylate | −28% | −19% | −11% | −9% |
| Citronellyl Prop | −1% | −5% | −5% | −7% |
| Cyclogalbanate | −89% | −85% | −78% | −70% |
| Eth Linalyl Acet i | 10% | −6% | −3% | −3% |
| Eth Linalyl Acet ii | −5% | −6% | −3% | −3% |
| Farnesyl Acet i | −35% | −23% | −19% | −14% |
| Farnesyl Acet ii | −36% | −23% | −13% | −8% |
| Geranyl Acet | −41% | −26% | −16% | −13% |
| Geranyl Iso Buty | −11% | −8% | −5% | −5% |
| Geranyl Prop | −26% | −16% | −10% | −9% |
| Geranyl Tiglate | −7% | −7% | −4% | −5% |
| Linalyl Acet | −5% | −7% | −2% | −2% |
| Linalyl Iso Buty | 0% | −3% | 0% | −2% |
| Neryl Acet | −35% | −22% | −13% | −11% |
| Pinocarvyl Acet | −8% | −8% | −3% | −3% |
| Prenyl Acet | −41% | −25% | −12% | −9% |

THIRD FRAGRANCE ACCORD

| | | | | |
|---|---|---|---|---|
| Amyl Sal | −51% | −30% | −17% | 8% |
| Celeriax i | −100% | −100% | −98% | 86% |
| Celeriax ii | −100% | −100% | −100% | −100% |
| Cis-3-Hexenyl Sal | −63% | −42% | −25% | −13% |
| Coumarin | −7% | −6% | −5% | −2% |
| Cresyl Acet | −100% | −100% | −100% | −100% |
| Cresyl Iso Buty | −100% | −100% | −100% | −100% |
| Cyclohexyl Sal | −12% | −5% | −6% | −3% |
| Eth Sal | −60% | −38% | −22% | −11% |
| Hexyl Sal | −49% | −29% | −17% | −8% |
| Honey Fi | −100% | −100% | −100% | −100% |
| Honey Fii | −100% | −100% | −100% | −100% |
| Iso Amyl Sal | −25% | −1% | 6% | 9% |
| Iso Butavan | −100% | −100% | −100% | −100% |
| Maltol Iso Buty | −100% | −100% | −100% | −90% |
| Meth Anisate | −17% | −10% | −7% | −3% |
| Meth Sal | −65% | −66% | −50% | −31% |
| Oceanol | −14% | −7% | −6% | 0% |

Stability of the dihydrocuminyl acetate and cinnamyl acetate present in the second fragrance accord was evaluated by detecting alcohol release by GC-MS. Results are shown in TABLE E8 below. The 2,2′-(alkylimino)bis[ethanol] tested is denoted by the alkyl chain length (C1, C4, C8, C12) and control is denoted by “Control” in the table.

TABLE E8
Alcohol release (mean ± standard deviation) measure by GC-MS
for test and control samples of the second fragrance accord.

| | Con- | | | | |
|---|---|---|---|---|---|
| | trol | C1 | C4 | C8 | C12 |
| Cinnamyl | Not | 1.18E+07 ± | 7.51E+06 ± | 5.13E+06 ± | 3.70E+06 ± |
| Alc | detec- | 6.82E+05 | 2.91E+05 | 2.40E+05 | 6.17E+05 |
| | ted | | | | |
| Dihydro | Not | 7.77E+06 ± | 4.34E+06 ± | 2.78E+06 ± | 1.95E+06 ± |
| Cuminyl | detec- | 3.78E+05 | 1.70E+05 | 5.51E+04 | 2.81E+05 |
| Alc | ted | | | | |

These results suggest that the stability of esters is affected at least in part by the 2,2′-(alkylimino)bis[ethanol] alkyl chain length, with certain lengths having more preferable stability outcomes for certain esters. These results are supportive of the use of 2,2′-(alkylimino)bis[ethanol] having specific alkyl chain lengths (e.g., C₄₋₁₂) in fragrance compositions containing select ester fragrance ingredients.

### Example VIII: Fabric Spray Application

The ability of 2,2′-(octylimino)bis[ethanol] (C₈ linear alkyl) to affect the sensory perception of sweat malodor was evaluated in a fabric refresher spray application using a sweat malodor model based on a proprietary formulation.

Fabric refresher spray samples were prepared by adding and mixing 2,2′-(octylimino)bis[ethanol] or fragrance, Floral HCA (high coverage accord), to a fabric refresher spray base (see, TABLE E9 for base composition) in a pump spray glass container. Floral HCA is a proprietary accord of fragrance ingredients that (1) can be dosed at a low concentration while still providing a high level of sweat malodor coverage and (2) has demonstrated at least an 80% reduction of sweat malodor in prior sensory testing.

TABLE E9
Fabric refresher spray base composition:

| | Approximate % |
|---|---|
| | Concentration in |
| INCI | Base Formula |
| Aqua | 88-88.5 |
| Alcohol | 10.0-10.1 |
| PEG-40 Hydrogenated Castor Oil | 0.9-1.1 |
| Fragrance or 2,2′-(octylimino)bis[ethanol] | 0.1-0.5 |

Samples for evaluation were prepared by spraying 1 pump (˜0.18 g) of sweat malodor onto a cloth substrate (3.5 in diameter). After 1 minute, the fabric refresher spray test sample was used to spray 1 pump (0.12 g) of product onto the same cloth substrate. The cloth substrate was transferred to a 16 oz. jar and immediately closed with an air-tight lid. After 4 hours, samples were presented in a blind and random order to 6 trained panelists. The panelists were instructed to take the steps of i) uncap the jar; ii) place their noses at a distance of about 2-3 inches above the opening; iii) take short sniffs for 3 seconds; and iv) enter a rating of malodor intensity on a scale of 0 (lowest) to 10 (highest).

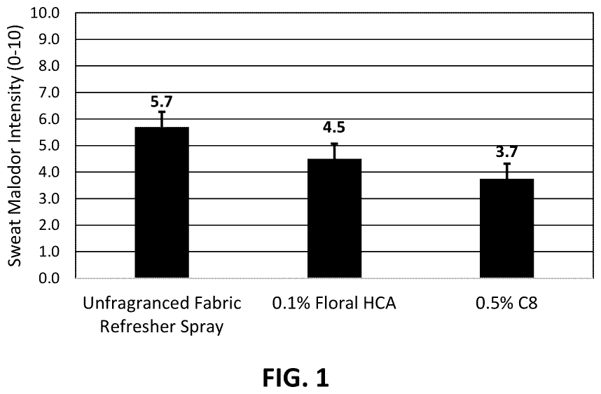

FIG. 1 is a bar chart showing the mean and standard deviation of the sweat malodor intensity rating provided by the panelists. As shown in FIG. 1, sweat malodor intensity ratings were lower when fabric spray including 2,2′-(octylimino)bis[ethanol] was used compared to sprays containing Flora HCA and unfragranced sprays.

These results are supportive of the ability of the 2,2′-(octylimino)bis[ethanol] to decrease sweat malodor intensity in cloth substrates.

### Example IX: Deodorant Application

The ability of 2,2′-(octylimino)bis[ethanol] to affect the sensory perception of sweat malodor was evaluated in-use with 33 subjects (19 men and 14 women between 20 and 50 years of age).

Test deodorants: Test deodorants: Two aerosol candidates were prepared as follow: an exemplary non-antiperspirant (non-AP) spray and the exemplary non-AP spray including 0.5% 2,2′-(octylimino)bis[ethanol].

Experimental Procedure: Experimental Procedure: For five days before the test sensory evaluation (i.e., the “test phase”), the subjects underwent a “wash-out phase” during which they substituted their normal bodywash with an unfragranced shower gel and were asked to not use any underarm product. On day 5 of the “wash-out phase,” subjects performed a workout session between 8 h and 12 h. The subjects were asked to rate the sweat malodor intensity of their axilla on a 0 (lowest) to 10 (highest) point scale before (at 8 h) and after (at T12 h) the workout session. The sensory results were used as a baseline.

On day 6 (“test phase”) subjects showered with the unfragranced shower gel and applied the 2 aerosol candidates (one to each axilla) through a standardized procedure with a blind and randomized (left/right) design. Each subject wore both candidates. The subjects performed a workout session between 8 h and 12 h. As with day 5 of the “wash-out phase,” the subjects rated the sweat malodor intensity of their axilla on a 0-10 point scale before (8 h) and after (12 h) the workout session.

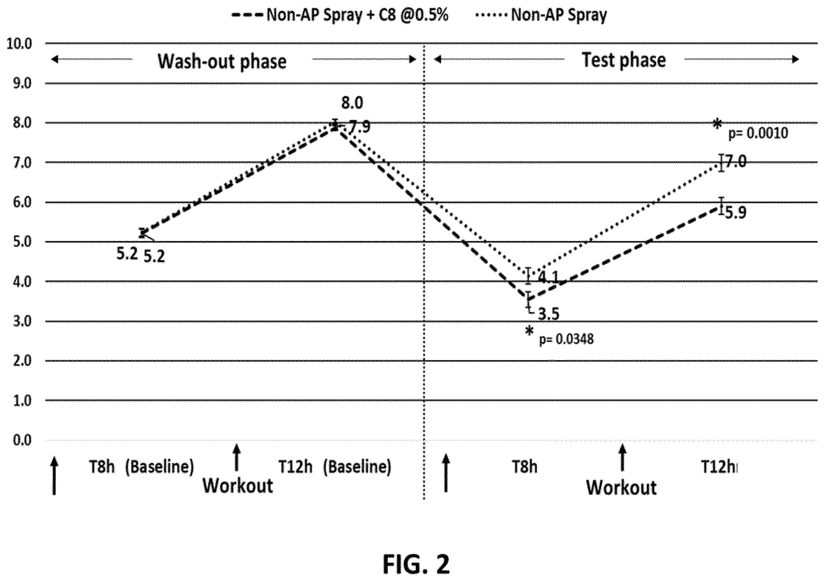

Results: FIG. 2 shows the mean and standard deviation of sweat malodor intensity ratings at 8 h and 12 h on day 5 of the “wash-out phase” and at 8 h and 12 h on day 6 (the “test phase”). As demonstrated in FIG. 2, sweat malodor intensity was reported as lower with the non-AP spray containing 2,2′-(octylimino)bis[ethanol] compared to the candidate lacking 2,2′-(octylimino)bis[ethanol] 8 h p=0.0348; 12 h p=0.0010).

These results suggest the ability of the 2,2′-(octylimino)bis[ethanol] to decrease the sweat malodor intensity when applied to human tissue.

### Example X: Body Spray

A model body spray base was formulated to contain 1% isopropyl myristate and 48% ethanol (190 proof). An isovaleric acid malodor was prepared. A dilution of 2,2′-(octylimino)bis[ethanol] was prepared, 25% w/w in ethanol (190 proof). Concentrated hydrochloric acid was used as received (37% w/w in water). Test and control samples were prepared in triplicate in 20 mL VOC vials.

TABLE E10
Model body spray sample formulation:

| | Body | | 37% | Isovaleric |
|---|---|---|---|---|
| | Spray | 2,2′-(octylimino) | hydrochloric | acid |
| Sample | Base | bis[ethanol] | acid | malodor |
| Control | 960 mg | none | none | 40 mg |
| 0.5% w/w 2,2′- | 938 mg | 20 mg (25% | 2.3 mg | 40 mg |
| (octylimino) | | w/w dilution) | | |
| bis[ethanol] | | | | |
| 2% w/w 2,2′- | 931 mg | 20 mg | 9.1 mg | 40 mg |
| (octylimino) | | (undiluted) | | |
| bis[ethanol] | | | | |

Each sample was closed immediately with a cap containing PTFE faced 0.125″ silicone septum, and mixed. Each vial was analyzed by piercing with a needle and passing headspace through a Gerstel thermal desorption tube packed with Tenax-TA adsorbent, using a pump at 50 mL/min for 2 min. Tubes were desorbed on a thermal desorption unit connected to GC-MS for peak identification and quantification.

The results shown in the table below demonstrate that 2,2′-(octylimino)bis[ethanol] treated with stoichiometric amounts of hydrochloric acid were effective in lowering the headspace concentration of isovaleric acid relative to the control sample.

TABLE E11
Isovaleric acid (mean ± standard deviation) measure by GC-MS
for control and body spray test samples.

| | Peak area, | | |
|---|---|---|---|
| | average of | | Change |
| | three | Standard | from |
| | replicates | deviation | control |
| Control | 22832.79 | 2448.481 | N/A |
| 0.5% 2,2′- | 5396 | 1061.631 | −76% |
| (octylimino)bis[ethanol] + | | | |
| HCl | | | |
| 2.0% 2,2′- | 1693 | 627.6469 | −93% |
| (octylimino)bis[ethanol] + | | | |
| HCl | | | |

These results support the ability of 2,2′-(octylimino)bis[ethanol] mixed with an acid agent to counteract malodor.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. Although the invention may be described in connection with specific preferred embodiments, it should be understood that the invention as claimed should not be unduly limited to such specific embodiments. Indeed, various modifications of the described modes for carrying out the invention which are obvious to those skilled in chemistry, perfumery, or related fields are intended to be within the scope of the following claims.

What is claimed is:

1. A method of counteracting sweat malodor in an air space or a substrate comprising the step of introducing a composition into the air space or the substrate, wherein the composition comprises 2,2′-(octylimino)bis[ethanol].

2. The method of claim 1, wherein the composition further comprises a fragrance compound selected from the group consisting of phenethyl alcohol, tetrahydrolinalool, linalool, 3-phenylpropanal, cinnamaldehyde, decanal, ethyl linalool, dihydromyrcenol, 2-methyldecanal, Veridian, undecavertol, and a mixture thereof.

3. The method of claim 2, wherein 2,2′-(octylimino)bis[ethanol] and the fragrance compound have a weight ratio of at least 1:1.

4. The method of claim 3, wherein the weight ratio is from 2:1 to 4:1.

5. A composition for counteracting sweat malodor in air space or a substrate comprising 2,2′-(octylimino)bis[ethanol].

6. The composition of claim 5 further comprising a fragrance compound selected from the group consisting of phenethyl alcohol, tetrahydrolinalool, linalool, 3-phenylpropanal, cinnamaldehyde, decanal, ethyl linalool, dihydromyrcenol, 2-methyldecanal, Veridian, undecavertol and a mixture thereof.

7. A functional product comprising a composition for counteracting sweat malodor in air space or a substrate, wherein the composition comprises 2,2′-(octylimino)bis[ethanol].

8. The functional product of claim 7, wherein the function product is selected from the group consisting of a room freshener spray, a fragrance diffuser, a candle, a sachet, a clothes deodorant, a detergent, a fabric softener, a fabric refresher, a linen spray, a disposable diaper, a diaper pail deodorant, an antiperspirant, a deodorant, a garbage bag, a car freshener, a pet care product, and an animal litter material.

9. The functional product of claim 7, wherein the composition further comprises a fragrance compound selected from the group consisting of phenethyl alcohol, tetrahydrolinalool, linalool, 3-phenylpropanal, cinnamaldehyde, decanal, ethyl linalool, dihydromyrcenol, 2-methyldecanal, Veridian, undecavertol, and a mixture thereof.

10. The fragrance composition of claim 5 further comprising a polymer.

11. The fragrance formulation of claim 10, wherein the polymer is selected from the group consisting of polyacrylate, polyurea, polyurethane, polyacrylamide, polyester, polyether, polyamide, poly(acrylate-co-acrylamide), starch, silica, gelatin and gum Arabic, alginate, chitosan, polylactide, poly(melamine-formaldehyde), poly(urea-formaldehyde) and a combination thereof.
